(19) United States
(12) Patent Application Publication
(10) Pub. No.: US 2010/0228060 A1
(43) Pub. Date: Sep. 9, 2010

(54) PERFLUORO-ARYLIODONIUM SALTS IN NUCLEOPHILIC AROMATIC 18F-FLUORINATION

(76) Inventors: Bengt Langstrom, Uppsala (SE); Farhad Karimi, Mansfield, MA (US)

Correspondence Address: GE HEALTHCARE, INC., IP DEPARTMENT 101 CARNEGIE CENTER, PRINCETON, NJ, 08540-6231, US

(21) Appl. No.: 12/681,629

(22) PCT Filed: Oct. 2, 2008
(86) PCT No.: PCT/US08/78547

§ 371 Date: Apr. 5, 2010

Related U.S. Application Data
(60) Provisional application No. 60/977,108, filed on Oct. 3, 2007.

(51) Int. Cl. C07C 17/20

(52) U.S. Cl. 570/147

(57) ABSTRACT

The present invention describes using fluorous chemistry in n.c.a. nucleophilic aromatic 18F-fluorination reactions by using perfluoro-aryliodonium salts as a precursor for aromatic nucleophilic substitution using a [18F] F-anion to displace a suitable leaving group from an electron deficient benzene ring. The results showed that using perfluoro-aryliodonium salts as a precursor is a suitable leaving group for n. c. a. nucleophilic aromatic 18F-fluorination in synthesis. The PT-precursor seems to be quite stable. In an attempt to purify the crude 18F-labeled product using fluorous solid phase extraction (F-SPE), the radio labeled impurities decreased significantly. Thus, it is possible to use this PT methodology to simplify and speed up purification methods.

### FIELD OF THE INVENTION

The present invention describes using fluorous chemistry in n.c.a. nucleophilic aromatic ¹⁸F -fluorination reactions by using perfluoro-aryliodonium salts as a precursor for aromatic nucleophilic substitution using an [¹⁸F] F-anion to displace a suitable leaving group from an electron deficient benzene ring. The present invention further relates to radiopharmaceutical kits for the preparation of aryl fluorides from diaryliodonium salts and fluoride ions in acetonitrile. The present invention additionally presents a method of use for preparing aryl fluorides and similar compounds thereof by using fast F-SPE. The present invention further presents a use of the process for manufacturing aryl fluorides and similar compounds thereof by using fast F-SPE.

### BACKGROUND OF THE INVENTION

Positron emission tomography (“PET”) is a non-invasive imaging technique which allows in vivo measurements and quantification of biological and biochemical process at the molecular level, and thus it is considered as a Molecular Imaging technique. Czermin J and Phelps M. Annu Rev Med 2002; 53: 89-112. PET is not only a valuable diagnostic tool in oncology, cardiology and neurology but is also becoming a valuable tool in nuclear medicine for drug development. Id. There are a number of positron emitting radionuclides of interest, such as ¹⁵O, ¹³N, ¹¹C, ¹⁸F, ⁷⁶Br, ¹²⁴I and metals like ⁶⁸Ga, ⁶⁹Cu and ⁶⁴Cu. They all have properties of interest for various applications, especially ¹¹C, ¹⁸F and the other halogens are of interest because of their properties in a synthetic labeling perspective. Additionally, ¹⁸F is of interest due to its physical properties. There are also a number of drugs containing one or more fluorine atoms. In some studies within drug development the need of specific radioactivity is less, for example in straightforward distribution studies, so in these cases F-exchange could be used as the labeling method.

In general, fluorine is a small atom with a very high electronegativity. Id. Covalently bound fluorine is larger than a hydrogen atom but occupying a smaller van der Waal's volume than a methyl, amino or hydroxyl group. Id. Fluorine substituent effects on pharmacokinetics and pharmacodynamics are very obvious. Eckelman W C. Nucl Med Bio 2002; 29: 777-782. Therefore, the replacement of a hydrogen atom or a hydroxy group by a fluorine atom is a strategy frequently applied in both PET tracer and drug developments. Id. The replacement of a hydrogen atom by a fluorine atom can alter the pKa, the dipole moments, lipophilicity, hydrogen bonding, the chemical reactivity, the oxidative stability, the chemical reactivity of neighboring groups or metabolic processes. Smart B. E. J Fluorine Chemistry 2001; 109: 3-11. The replacement of a hydroxyl group is based on the hypothesis that fluorine is a hydrogen acceptor like the oxygen of a hydroxyl group. Czermin J and Phelps M. Annu Rev Med 2002; 53: 89-112.

As regards of its use for PET, fluorine-18 has excellent nuclear properties such as low positron energy that results in low radiation dose, short maximum range in tissue and convenient half-life (t_{1/2}=109.7 min) considering distribution to other hospitals and performing longer acquisition protocols.

Furthermore, the application of radiolabelled bioactive peptides for diagnostic imaging is gaining importance in nuclear medicine. Biologically active molecules, which selectively interact with specific cell types, are useful for the delivery of radioactivity to target tissues. For example, radiolabelled peptides have significant potential for the delivery of radionuclides to tumours, infarcts, and infected tissues for diagnostic imaging and radiotherapy. ¹⁸F is the positron-emitting nuclide of choice for many receptor-imaging studies. Therefore, ¹⁸F-labelled bioactive peptides have great clinical potential because of their utility in PET to quantitatively detect and characterise a wide variety of diseases.

Radiolabeling of compounds with [¹⁸F]-fluoride can be achieved either by indirect displacement using fluoroalkylation agents or direct displacement of a leaving group. Using fluoroalkylation agents or direct displacement is not always convenient for all pharmaceutical substrates due to the formation of by-products, low yield, and the difficulties in purification processes.

Therefore, the aim of this invention is to develop fluorous chemistry also known as ponytail chemistry, (“PT”) in a no carrier added (“n.c.a.”) nucleophilic aromatic ¹⁸F-fluorination reactions by using perfluoro-aryliodonium salts as a precursor for aromatic nucleophilic substitution using an [¹⁸F] F-anion to displace a suitable leaving group from an electron deficient benzene ring. This process offers simplifications of the overall process going from [¹⁸F]-fluoride in target water to pure radio-pharmaceutical since the compounds containing the ponytail can easily be removed by solid-phase extraction (“SPE”)-purification where the SPE-matrix contains a ponytail matrix.

Discussion or citation of a reference herein shall not be construed as an admission that such reference is prior art to the present invention.

### SUMMARY OF THE INVENTION

Fluorinated compounds are synthesized in pharmaceutical research on a routine basis and many marketed compounds contain fluorine. Quite often, fluorine is introduced to improve the metabolic stability by blocking metabolically labile sites. However, fluorine can also be used to modulate the physicochemical properties, such as lipophilicity or basicity. Fluorine has been used to enhance the binding affinity to certain target proteins.

Diaryliodonium salts have been shown to react with a fluoride ion at a temperature of about 40° C. to about 130° C. in acetonitrile to generate aryl fluorides. The perfluoro-aryliodonium salt ponytail (“PT”)-precursor seems to be quite stable for at least 4-6 months. In an attempt to purify the crude aromatic ¹⁸F-labeled product using fluoride-solid phase extraction (“F-SPE”), the radio labeled impurities decreased significantly by about 80%.

The present invention investigates the use of diaryliodonium salts as a suitable precursor for aromatic nucleophilic substitution using an [¹⁸F] F-anion to displace a suitable leaving group from an electron deficient benzene ring.

One embodiment of the present invention encompasses a method for radiofluorination comprising a reaction of the following compounds:

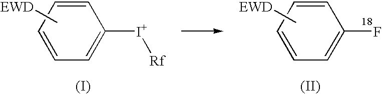

wherein Rf is a polyfluorinated alkyl or aryl compound and the diaryliodonium salts react with fluoride ions placed in acetonitrile at a temperature of about 40° C. to about 130° C. thereby generating compound (II) and then whereby compound (II) is purified using SPE, solid phase extraction.

### DETAILED DESCRIPTION OF THE INVENTION

Fluorous compounds contain a perfluoroalkyl group and virtually any molecule can have a fluorous analog. The perfluoroalkyl chain remains chemically inert during the reaction, while imparting unique properties to the reagents and sorbents during separation. These properties are due to a highly selective affinity (fluorous affinity interaction) between the reagent fluorous groups and the sorbent fluorous groups.

During separation, the chromatographic properties of the perfluoroalkyl group dominate the molecule's other functional groups. This critical property makes the organic domains of the fluorous molecules become chromatographically irrelevant to the fluorous sorbent. Hence the immense benefit of fluorous technology is that diverse chemical structures containing the same fluorous group can be purified by simply using a single chromatographic method.

Fluorous Solid Phase Extraction (“F-SPE”) quickly separates fluorous compounds from non-fluorous compounds in three easy steps. First, the reaction mixture is loaded onto the column. Second, the non-fluorous compounds are eluted with a fluorophobic solvent in one fraction. Third, the fluorous compounds are eluted with a fluorophilic solvent.

Furthermore, fluorous substrates are used to deliver a product that contains a fluorous tag. SPE can then be used to recover the individual, highly pure fluorous product from non-fluorous reagents. In the reverse approach, fluorous reagents can be used such that the byproducts are fluorous while the desired product is non-fluorous. Simple separation by F-SPE yields a high purity product.

The aim of the present invention is to develop fluorous chemistry, also known as ponytail (“PT”) chemistry, in a n.c.a. nucleophilic aromatic ¹⁸F-fluorination reaction by using perfluoro-aryliodonium salts as a precursor for aromatic nucleophilic substitution and using an [¹⁸F] F-anion to displace a suitable leaving group from an electron deficient benzene ring. Using PT chemistry offers potential simplifications of the overall process going from [¹⁸F]-fluoride in target water to pure radio-pharmaceutical since the compounds containing the ponytail easily can be removed and the product purified using solid phase extraction (“SPE”) where the SPE contains a ponytail matrix. A ponytail matrix is defined herein as a polyfluorinated compound such as a polyfluorinated alkyl chain or polyfluorinated aryl moiety.

There are various advantages of using a solid phase extraction approach over conventional liquid synthesis approaches in labeling reactions.

One advantage in using a solid phase approach over conventional liquid synthesis in labeling reactions is the simplified kit-concept of using the solid phase approach i.e. direct ¹⁸F fluorination reactions. Another advantage is the easy cleanup in between consecutive reaction steps using the solid phase approach. Yet one other advantage of using the solid phase approach is the improved purification the solid phase approach delivers in labeling reactions in comparison. Still a further advantage of the present invention presents that the solid phase approach has a much easier automated process in comparison to the conventional liquid synthesis. Another advantage of the present invention's use of a solid phase approach depicts an improved yield of product through a time optimized process that is in comparison to other conventional synthesis.

One embodiment of the present invention encompasses a method for radiofluorination comprising a reaction of the following compounds:

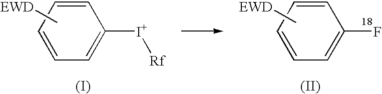

wherein Rf is a polyfluorinated alkyl or aryl compound and the diaryliodonium salts react with fluoride ions placed in acetonitrile at a temperature of about 40° C. to about 130° C. thereby generating compound (II) and then whereby compound (II) is purified using SPE.

Another embodiment of the present invention shows that the SPE contains a ponytail matrix and the SPE occurs at least twice as fast as conventional liquid synthesis processes.

A further embodiment of the present invention depicts the temperature at which diaryliodonium salts have been shown to react with fluoride ions placed in acetonitrile is about 50° C. to about 110° C. More preferably, the temperature is about 80° C.

Still another embodiment of the present invention shows a radiopharmaceutical kit for preparing a compound of formula (II) and similar compounds thereof.

An additional embodiment of the present invention depicts a method for the use of preparing a compound of formula (II).

Yet another embodiment of the present invention shows the use of the process for manufacturing a compound of formula (II).

### EXAMPLES

The invention is further described in the following examples, which is in no way intended to limit the scope of the invention.

### Precursor Synthesis

The precursor synthesis used in this invention was obtained using Scheme 1 below. A perfluoro-aryliodonium salt is used as a precursor for aromatic nucleophilic substitution in an [¹⁸F] F-anion to displace a suitable leaving group from an electron deficient benzene ring.

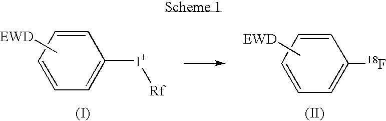

Using PT chemistry offers potential simplifications of the overall process going from [¹⁸F]-fluoride in target water to pure radio-pharmaceutical since the compounds containing the ponytail easily can be removed and the product purified using solid phase extraction (“SPE”) where the SPE contains a ponytail matrix.

### ¹⁸F Production

[¹⁸F] Fluoride was produced at Uppsala Imanet by an ¹⁸O (proton, neutron) ¹⁸F nuclear reaction through proton irradiation of enriched (95%) ¹⁸O water using Scanditronix MC-17 cyclotron.

### Method for Preparing Aromatic Nucleophilic Substitution ¹⁸F-Labeling Using Perfluoro-Aryliodonium Salts

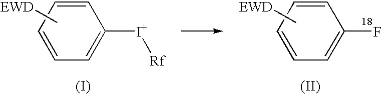

A solution of (I) containing (5.0 milligrams) in 0.2 milliliter of acetonitrile at a temperature in the range of about 50° Celsius to about 110° Celsius was added to fluoride ions to generate aryl fluorides, (II). The reaction was performed in a closed vessel for about 15 minutes.

The results using precursor II, containing perfluoro-aryliodonium salts, showed that this is one suitable leaving group for n. c. a. nucleophilic aromatic ¹⁸F-fluorination reaction. The possibilities for fluorous SPE purification methods was illustrated using Fluor° Flash® which in using this example gave a substantial purification of the labeled product.

Furthermore, the solid phase extraction is applicable in essentially all areas from traditional synthesis through parallel synthesis, and is especially useful for parallel synthesis of intermediates.

The PT-precursor seems to be stable for at least 4-6 months. New PT-precursors should be synthesized for exploring the scope and limitation of this methodology. This example depicts using suitable perfluoro-substituted leaving groups and combining them with fast Fluorous SPE purification approaches.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for radiofluorination comprising a reaction of the following compounds: 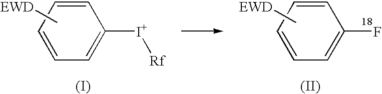
wherein Rf, a polyfluorinated alkyl or aryl compound, and the diaryliodonium salts react with fluoride ions placed in acetonitrile at a temperature of about 40° C. to about 130° C. thereby generating compound (II) and then whereby compound (II) is purified using SPE.

2. The method according to claim 1, wherein the SPE contains a ponytail matrix.

3. The method according to claim 1, wherein the SPE occurs at least twice as fast as conventional liquid synthesis processes.

4. The method according to claim 1, wherein the temperature is preferably about 50° C. to about 110° C.

5. The method according to claim 1, wherein the temperature is more preferably about 80° C.

6. A radiopharmaceutical kit for preparing a compound of formula (II) according to claim 1.

7. A method for the use of preparing a compound of formula (II) according to claim 1.

8. A use of the process for manufacturing a compound of formula (II), according to claim 1.
